(19) United States
(12) Patent Application Publication
(10) Pub. No.: US 2004/0248289 A1
(43) Pub. Date: Dec. 9, 2004

(54) Tandem repeat markers

(76) Inventors: Vladimir Noskov, Rockville, MD (US); Vladimir Larionov, Potomac, MD (US); Natalay Kouprina, Potomac, MD (US); J. Carl Barrett, Rockville, MD (US)

Correspondence Address: NATIONAL INSTITUTE OF HEALTH, C/O NEEDLE & ROSENBERG, P.C., SUITE 1000, 999 PEACHTREE STREET, ATLANTA, GA, 30303, US

(21) Appl. No.: 10/491,774

(22) PCT Filed: Oct. 4, 2002
(86) PCT No.: PCT/US02/31634

(30) Foreign Application Priority Data
Oct. 4, 2001 (US) 60327189

(51) Int. Cl. C12N 15/00

(52) U.S. Cl. 435/320.1

(57) ABSTRACT

Disclosed is a vector comprising a marker sequence, a hook capable of attaching the vector to a target molecule, and a vector diagnostic sequence having the same sequence as a target diagnostic sequence contained in the target molecule, wherein the marker, hook, and vector diagnostic sequence are arranged (5′) to (3′) such that the marker would be in between the vector diagnostic sequence and the target diagnostic sequence after the vector and the target molecule are attached via the hook. Also provided is a method of attaching two nucleic acid molecules together, comprising mixing a target molecule and the vector together under conditions that promote the attachment of the target molecule and the vector.

This application claims priority to U.S. Provisional Application No. 60/327,189 filed on Oct. 4, 2001, entitled “Tandem Repeat Markers,” which application is herein incorporated by reference in its entirety.

### I. BACKGROUND

Yeast Artificial Chromosome (YAC) and Bacterial Artificial Chromosome (13AC) cloning systems have greatly facilitated the analysis and understanding of complex genomes (1, 2). These techniques male it possible to isolate large DNA fragments, thereby greatly simplifying the physical mapping of chromosomes and genomes. However, the process of isolating a gene or specific chromosomal region of interest is labor-intensive, requiring characterization of thousands of YAC or BAC clones and time consuming sub-cloning procedures. In addition, different regions of the same gene are often on different YACs or BACs, requiring multiple cloning steps to reassemble a copy of the gene. For cloning DNA from the genome of a particular individual, a library must be constructed specifically for that purpose, and standard YAC or BAC cloning strategies are not suitable for genomic regions in which rearrangements have occurred.

Recently, a recombinational cloning strategy was developed that allows genes and chromosomal regions to be isolated from a complex genome without prior construction of a genomic DNA library (3, 4). This technique is carried out in yeast cells, which have a high level of homologous recombination. Botstein and colleagues (5) who showed that a double-strand DNA break is efficiently repaired when it is cotransformed into yeast with a linear DNA fragment that includes DNA sequence that is both 5′ and 3′ to the double-strand DNA break. The in vivo homologous recombination pathway that joins together two different DNA fragments sharing homology is now routinely used for construction of recombinant plasmids (6-8).

The TAR cloning methods described above allow a gene to be isolated directly from total genomic DNA; however, these methods have a relatively high background rate of recombination. End-joining and non homologous recombination between the vector and genomic DNA generate YAC clones that propagate in yeast even though they do not carry the gene of interest. Typically, TAR cloning produces a set of clones in which the desired gene occurs at frequency of ˜0.5%. Clones carrying the gene of interest are usually identified by PCR or colony hybridization.

Disclosed is a selection system that can be used in TAR and in many other nucleic acid manipulation techniques. The selection system allows for higher specificity and lower background and can utilize positive and negative genetic selection for clones with the gene of interest. The desired gene can be selected from primary transformants with an efficiency close to 100%.

### II. SUMMARY

Disclosed herein are compositions and methods for selection of nucleic acids.

### III.BRIEF DESCRIPTION OF THE DRAWINGS

The accompanying drawings, which are incorporated in and constitute a part of this specification, illustrate several embodiments and together with the description, serve to explain that which is disclosed.

FIG. 1 shows a schematic diagram of genetic selection of gene-positive YAC clones. The TAR vector carries a yeast centromere (CEN6), a yeast positive selectable marker (HIS3), two gene-specific targeting hooks (or one gene-specific hook and one common repeat hook) and a negative-selectable marker (URA3). The TAR vector also contains a sequence called a VDS that is distal to the gene-specific targeting hook sequence in the targeted chromosomal region and proximal to URA3 and the gene-specific targeting hook in the TAR vector. (In the diagram, only the end of the TAR vector carrying the gene-specific targeting hook is shown.) A. Homologous recombination between the gene-specific targeting hook and a genomic fragment containing the gene of interest leads to duplication of the VDS in the YAC. The URA3 marker is flanked by a direct repeat of the VDS, which is mitotically unstable in yeast. Such clones can be easily detected by their ability to grow on media containing 5-fluoro-orotate (selects for Ura7⁻ phenotype). B. Non homologous recombination between a hook and a genomic fragment (or non-homologous end-joining) forms a YAC with one copy of the VDS. In these YACs, the URA3 marker is stable, and cells with these YACs do not grow on media containing 5-FO.

FIG. 2 shows a direct selection of gene-positive clones on 5-FO containing medium. Two Tg.AC transgene-positive and 23 transgene-negative transformants were replica plated on a 5-FO complete medium lacking histidine. Colonies containing the transgene YACs exhibit a papillae growth as a result of “pop-out” event of the URA3 marker. Between five and one hundred Ura7⁻ “pop-out” events were observed on replicas of gene-positive colonies. “Top-out” events are explained by generation of an unstable duplication of a VDS in the gene-positive YAC clones as predicted from the scheme (see FIG. 1A).

FIG. 3 shows a molecular analysis of background clones. The ends of 44 randomly selected background YACs (lacking HPRI) obtained during HPRT cloning were rescued as plasmids in E. coli. Terminal sequences of the YAC inserts were determined. Thirty-eight clones (87%) have a non-rearranged 60 bp gene-specific hook sequence; these clones form by non-homologous end joining rather than by homologous recombination. Other clones have a partially deleted gene-specific targeting hook, and could form by degradation of the end of the hook followed by non-homologous end-joining or by homologous recombination.

FIG. 4 shows the positive transformants obtained using the disclosed vectors.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

In general, compositions and methods are disclosed which are related to nucleic acid selection and isolation. Vectors are disclosed that have the unique ability to remove a marker that is associated with the vector after the vector has attached, through for example, homologous recombination, with a target molecule. The removal of the marker occurs in a preferential manner, when the correct target molecule has been attached to the vector. If spurious or unintended attachment of the vector has occurred, the marker will almost never be removed. This provides a very easy means for determining if attachment, through for example, homologous recombination has occurred with the desired target molecule or with non-desired molecules, because observance of the loss of the marker indicates correct vector/target molecule attachment. Thus, in many embodiments the compositions can comprise nucleic acids and in certain embodiments the compositions can also included polypeptides.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

Where the term “at least” x appears and x is a number it is understood that only x and about only X are also disclosed. For example, the phrase “at least 30%” would also be understood as disclosing “30%.”

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation. “Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Disclosed are the components to be used to prepare the disclosed compositions as well as the compositions themselves. The components and/or compositions can be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular vector is disclosed and discussed and a number of modifications that can be made to a number of molecules including the vector are discussed, specifically contemplated is each and every combination and permutation of the vector and the modifications that are possible unless specifically indicated to the contrary. Thus, if a class of molecules A, B, and C are disclosed as well as a class of molecules D, E, and F and an example of a combination molecule, A-D is disclosed, then even if each combination is not individually recited each is individually and collectively contemplated meaning combinations A-E, A-F, B-D, B-E, B-F, C-D, C-E, and C-F are considered disclosed. Likewise, any subset or combination of these combinations is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Throughout this application, various publications are referenced. The disclosures of these publications in their entireties are hereby incorporated by reference into this application in order to more fully explain the disclosed compositions and methods. The references disclosed are also individually and specifically incorporated by reference herein for the material contained in them that is discussed in the sentence in which the reference is relied upon.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homolgy to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using using each of calculation methods (although, in practice, the different calculation methods will often result in different calculated homology percentages

2. Hybridization/selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as the level of homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is, for example, in 10 or 100 or 1000 fold excess. This type of assay can be performed under conditions where both the limiting and non-limiting nucleic acid are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation. For example, if the enzymatic manipulation is DNA extension, then selective hybridization conditions would be those under which at least about 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

B. Methods of Using the Compositions Disclosed are methods of using the disclosed compositions. Typically these methods can be performed in any eulcaryotic or prokaryotic organism that can support homologous recombination. The disclosed vectors are designed to support both the attachment of the vector to a target molecule, such as through homologous recombination, and then after attachment the vectors can support an internal homologous recombination event that occurs in the product, if the vector is attached to the correct target molecule. This homologous recombination event that occurs in the product causes the removal of a detectable (e.g., visible, selectable, etc.) marker, and thus, those events that produced a correct product molecule can be separated from those events that do not.

The removal of the marker occurs in the correct product molecules because of the orientation of a sequence contained in the vector with the same or similar sequence contained in the target molecule. These sequences called a vector diagnostic sequence (VDS) and target diagnostic sequence (TDS) respectively form a tandem repeat in the product molecule, and tandem repeats, because of homologous recombination events, are unstable in organisms, particularly yeast. The recombination event that takes place between the TDS and the VDS, will among other things, remove the sequence that exists between the VDS and the TDS. If that sequence is the marker sequence, then the marker sequence will be removed during the recombination event. The VDS is chosen to correspond to a particular sequence contained within the target molecule, either natively or engineered into the target molecule. Thus, probabilities indicate that it is very unlikely that a non-target molecule will contain the appropriate sequence and that, because homologous recombination is dependent on among other things sequence similarity, the product molecules produced from the vector and the non-target molecule will not support recombination, and thus the marker will be maintained in the product molecule of undesired recombination events.

Disclosed are methods of attaching two nucleic acid molecules together comprising mixing a target molecule and the vector comprising a VDS together under conditions that promote the attachment of the target molecule and the vector.

Disclosed are methods, wherein the conditions are conditions that allow homologous recombination.

Disclosed are processes for making the compositions as well as making the intermediates leading to the compositions. For example, disclosed are vectors containing a VDS and a marker. There are a variety of methods that can be used for making these compositions, such as synthetic chemical methods and standard molecular biology methods. It is understood that the methods of making these and the other disclosed compositions are specifically disclosed. Also disclosed are processes for making cells comprising the disclosed nucleic acids, for making peptides related to the disclosed nucleic acids, and animals comprising any disclosed nucleic acid, peptide, or cell.

Disclosed are nucleic acids produced by the process of linking together a VDS and a marker such that when the nucleic acid interacts with a desired target molecule, the marker will be removed.

Disclosed are cells produced by the process of transforming the cell with any of the disclosed nucleic acids.

Disclosed are any of the disclosed peptides produced by the process of expressing any of the disclosed nucleic acids.

Disclosed are animals produced by the process of transfecting a cell within the animal with any of the nucleic acid molecules disclosed herein. Disclosed are animals produced by the process of transfecting a cell within the animal with any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclosed are animals produced by the process of adding to the animal any of the cells disclosed herein.

Disclosed are compositions and methods which can be used for the isolation of mutant forms of genes which are derived from a subject, such as a patient or a subject with a particular disease. These methods can be used to identify mutations that lead to the disease. Also disclosed are methods and compositions which can be used in th separation of haplotypes, i.e. long-range haplotyping. The compositions and methods can also be used to isolate gene homologs and orthologs.

1. Methods of Cloning and Nucleic Acid Manipulation

The disclosed vectors can be used in any cloning or nucleic acid manipulation procedure that occurs in eukaryotic or prokaryotic organism. For example, the vectors can be used in recombination cloning procedures, such as transformation associated recombination (TAR) procedures, including TAR cloning procedures.

In recombination cloning, vectors are introduced into a yeast which are designed to homologously recombine with a target molecule to form either a circular or linear YAC. Thus, in a recombination cloning procedure the product molecule contains at least, a yeast centromere, telomere, and yeast autonomous replication sequence (ARS). In general there are two types of recombination cloning procedures: 1) TAR procedures that utilize endogenous ARS sequences present in the target molecules and 2) basic recombination procedures that utilize vectors that contain an ARS. The TAR procedures are particular efficient for cloning from large libraries as only molecules which recombine with a target molecule can be propagated. The methods and variations disclosed in Larionov Vladimir, et al., “Direct isolation of human BRCA2 gene by transformation-associated recombination in yeast”, Proc. Natl. Acad, Sci., USA, vol. 94, pp. 7384-7387, July 1997; Larionov, Vladimir, et al., “Specific cloning of human DNA as yeast artificial chromosomes by transformation-associates recombination”, Proc. Natl. Acad. Sci., USA, vol. 93. , pp. 491-496, Janurary 1996; 4; Larionov, V., et al., Recombination during transformation as a source of chimeric mammalian artificial chromosomes in yeast (YACs), Nucleic Acids Research, vol. 22, No. 20, pp. 4154-4162, 1994; Kouprina, N., et al., “A Model System to Assess the Integrity of Mammalian YACs during Transformation and Propagation in Yeast”, Genomics. 21, pp. 7-17, 199; Larionov, V., Kouprina, N., Graves, J., and Resnick, M. A. (1996). Highly selective isolation of human DNAs from rodent-human hybrid cells as circular yeast artificial chromosomes by transformation-associated cloning, Proceedings of the National Academy of Sciences of the United States of America 93, pp. 13925-30; Humble M C, Kouprina N, Noskov V N, Graves J, Garner E, Tennant R W, Resnick M A, Larionov V, Cannon R E, Radial transformation-associated recombination cloning from the mouse genome: isolation of Tg.AC transgene with flanking DNAs, Genomics. 2000 Dec. 15;70(3):292-9; Kouprina N, Nikolaishvili N, Graves J, Koriabine M, Resnick M A, Larionov V, Integrity of human YACs during propagation in recombination-deficient yeast strains, Genomics. 1999 Mar. 15;56(3):262-73, Kouprina N, Campbell M, Graves J, Campbell E, Meincke L, Tesmer J, Grady D L, Doggett N A, Moyzis R K, Deaven L L, Larionov V, Construction of human chromosome 16- and 5-specific circular YAC/BAC libraries by in vivo recombination in yeast (TAR cloning), Genomics. 1998 Oct 1;53(1):21-8, Cancilla M R, Tainton K M, Barry A E, Larionov V, Kouprina N, Resnick M A, Sart D D, Choo, Direct cloning of human 10q25 neocentromere DNA using transformation-associated recombination (TAR) in yeast, Genomics. 1998 Feb. 1;47(3):399-404, Kouprina N, Eldarov M, Moyzis R, Resnick M, Larionov V., A model system to assess the integrity of mammalian YACs during transformation and propagation in yeast, Genomics. 1994 May 1;21(1):7-17; Prado, F., and Aguilera, A. (1994). New in-vivo cloning methods by homologous recombination in yeast.Current Genetics 25, pp. 180-3; Spencer, F., Ketner, G., Connelly, C., and Hieter, P. (1993), Targeted recombination-based cloning and manipulation of large DNA segments in yeast. Methods: A companion to Methods Enzymol 5, pp. 161-175; Bradshaw, Suzanne, M., et al., “A long-range regulatory element of Hoxc8 identified by using the pClasper vector”, Proc. Natl. Acad. Sci., USA, vol. 93, pp. 2426-2430, March 1996; Bradshaw, Suzanne M., et al. “A new vector for recombination-based cloning of large DNA fragments from yeast artificial chromosomes”, Nucleic Acids Research, vol. 23, No. 23, pp. 4850-4856, 1995; Bradshaw, Suzanne M., et al., “Site Specific Recombination-Mediated Isolation of a Large Sub-Region from a Mouse Hox-c YAC”, Ist Euro. Sci Found. Conf. On Deve. Bio., Karause Ittengen, Switzerland, Jun. 14-17, 1993; Ketner, Gary, et al., “Efficient manipulation of the human adenovirus genome as an infectious yeast artificial chromosome clone”, Proc. Natl. Acad. Sci., USA, vol. 91, pp. 6186-6190, June 1994; and Degryse, E., Dumas, B., Dietrich, M., Laruelle, L., and Ascstetter, T. (1995), In vivo cloning by homologous recombination in yeast using a two-plasmid-based system. Yeast 11, pp. 629-40; U.S. Pat. No. 6,221,588 for “Yeast-bacteria shuttle vector”, and U.S. patent application Ser. No. 09/060023 for “Transformation associated recombination cloning” filed on Apr. 14, 1998, each of which is herein incorporated by reference for materials related to recombination cloning and the included variations.

a) Transformation Associated Recombination

The following are particular embodiments of the TAR procedures discussed above. TAR cloning typically uses vectors without an ARS element that do not replicate in yeast unless an ARS, or a functional equivalent of an ARS, is acquired by recombination with genomic DNA. ARS sequences are frequently and randomly distributed throughout all eukaryotic genomes (i.e., one ARS per 20-40 kb, on average. Thus, most mammalian chromosomal regions can be isolated by TAR-cloning in yeast using an ARS-less vector.

There are a number of different general TAR systems. For example, two schemes that have been developed and characterized are disclosed herein. If DNA sequence information is available from the 3′ and 5′ flanking regions of the gene of interest, the gene can be isolated using a vector with two short unique sequences that flank the gene. These hooks are cloned into the vector in such a way that the linear form of the vector releases the gene targeting sequences. The hooks can be as small as 30 bp. Hooks of 60 bp typically recombine with an efficiency as great as hooks that are larger when used in TAR procedures.

A modified version of TAR cloning, called radial TAR cloning, has also been developed. Radial TAR cloning also uses a vector with two targeting hooks; however, one hook is a unique sequence from the chromosomal region of interest and the other hook is a repeated sequence that occurs frequently and randomly in the genomic DNA (i.e., Alu repeats in human DNA or B1 repeats in mouse DNA). In the radial cloning method, a set of nested overlapping fragments is isolated that extend from the gene-specific targeting hook to different upstream or downstream Alu (B1) positions within the gene of interest. This approach increases the likelihood that a clone will be formed and isolated that includes an ARS-like sequence.

The amount of DNA damage (i.e., dsDNA breaks, etc.) in the genomic DNA used in a TAR cloning experiment will determine the size of inserts in the YAC clones obtained by TAR. TAR cloning requires physical manipulation of the DNA, which causes some DNA shearing; thus the upper size limit of YACs obtained by TAR cloning is typically 250 kb. Circular YACs of 250 kb or less can easily be retrofitted into BACs and transferred into Esherichia coli for further characterization. TAR cloning has been used with success to isolate several single copy genes and specific chromosomal regions from human and mouse DNA.

Components that may be require for forming a yeast artificial chromosome (YAC) from the vector and the target molecule or having the vector itself be a YAC, such as the yeast centromere and a yeast telomere, are well known to those skilled in the art. These nucleic acid entities have previously been used in the construction of yeast artificial chromosomes (YACs). For example, see Schlessinger, D. for a general discussion of various YAC construction which is herein incorporate by reference for the material related to YACs. (“Yeast artificial chromosomes: tools for mapping and analysis of complex genomes” Trends in Genetics 6:248-264 (1990)). Additionally, the vector may further comprise a replication origin (ARS, autonomously replicating sequence). Where the vector does not contain a replication origin, such ARS sequence or ARS-like sequence may originate from the nucleic acid which recombines with the vector and becomes part of the YAC, thereby conferring on the YAC the capacity for replication. Alternatively, an ARS sequence may be within both the vector and the nucleic acid which recombines with the vector and becomes part of the YAC.

Thus, vectors that are designed to be useful in TAR cloning in yeast will typically have a yeast centromere as well as yeast telomeres as well as for example, an ARS. At the very least, there will typically be an ARS associated with the product molecule even if the ARS is not associated with the VDS vector.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. (see for example 1). In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination events occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

The disclosed nucleic acids make the initial detection of the homologous transfection event, much easier to monitor and track. To modify a gene of interest “a modification sequence” is cloned into a TAR vector along with hooks and diagnostic sequence, VDS. “A gene modification” sequence can be for example a heterologous or synthetic regulatory sequence. Specificity of gene targeting can be detected on destabilization of a flanking sequence in transformants. For gene disruption gene specific targeting sequences (hooks) and a diagnostic sequence are cloned into a TAR vector and the vector obtained is used for transformation. With use of a diagnostic sequence gene disruption events can be selected on a high loss of a counterselectable marker resulted from duplication of a diagnostic sequence in a vector. Typically the hooks and direct repeat sequences can undergo homologous recombination events.

3. Vectors/Delivery of the Compositions to Cells

A number of different methods can be used for the introduction of the vectors into yeast, mammalian, or other eukaryotic or prokaryotic cells, for example, electroporation, lipofection and calcium phosphate precipitation. The compositions can also be delivered through a variety of nucleic acid delivery systems, direct transfer of genetic material, in but not limited to, plasmids, viral vectors, viral nucleic acids, phage nucleic acids, phages, cosmids, or via transfer of genetic material in cells or carriers such as cationic liposomes. Such methods are well known in the art and readily adaptable for with the vectors described herein. In certain cases, the methods will be modified to specifically function with large DNA molecules. Further, these methods can be used to target certain diseases and cell populations by using the targeting characteristics of the carrier. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

As used herein, plasmid or viral vectors are agents that transport the VDS containing vector into the cell without degradation and include a promoter yielding expression of the gene in the cells into which it is delivered. In some embodiments the delivery vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

c) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson,.Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes. For example, replicating and host-restricted non-replicating vaccinia virus vectors.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro. For example, a preferred mode of delivery for in vivo uses would be the use of liposomes. Lipofection has yielded ˜5×10⁻⁵ neomycin-resistant transfectants per microgram of BAC/YAC DNA. The efficiency was much lower using the other procedures.

Thus, the compositions can comprise, in addition to the disclosed VDS vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

As described above, the compositions can be administered in a pharmaceutically acceptable carrier and can be delivered to the subject§s cells ill vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

It is understood that the disclosed vectors can be used in any type of cell that will allow homologous recombination to take place. For example, the disclosed vectors can be used in and delivered to mammalian cells, avian cells, and yeast cells as well as other eukaryotic and prokaryotic cells.

The disclosed vectors can be used and manipulated both in vitro and in vivo using the compositions and methods disclosed herein as well as those compositions and methods understood by the skilled artisan.

4. Pharmaceutical Carriers/Delivery of Pharamceutical Products

As described elsewhere herein, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447451, (1991); Bagshawe, K. D., Br. J. Cancer 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

a) Pharmaceutically Acceptable Carriers

The compositions can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

5. Uses as research tools

Other VDS vectors which do not have a specific pharmaceutical function, but which may be used for tracking changes within cellular chromosomes or for the delivery of diagnostic tools for example can be delivered in ways similar to those described for the pharmaceutical products.

The cloning vectors can be used for example as tools to isolate and study target sequences necessary for the completion, of large scale sequencing efforts, such as the Human Genome project.

The VDS vectors can also be used for example as tools to isolate and test new drug candidates for a variety of diseases. They can also be used for the continued isolation and study of, for example, the cell cycle

### C. Compositions

Disclosed are vectors that contain elements that when attached to a target molecule will cause the removal of a marker sequence contained in the vector sequence from the vector sequence.

Disclosed are vectors comprising a marker sequence, a hook capable of attaching the vector to a target molecule, and a sequence capable of recombining with a sequence in the target molecule producing a product molecule, wherein the marker is removed from the product molecule.

Disclosed are vectors comprising a marker sequence, a hook capable of attaching the vector to a target molecule producing a product molecule, and a vector diagnostic sequence capable of recombining with a sequence in the target molecule, wherein the marker is removed from the product molecule after the vector diagnostic sequence recombines with the target molecule.

Disclosed are vectors comprising a marker sequence, a hook capable of attaching the vector to a target molecule producing a product molecule, and a vector diagnostic sequence having the same sequence as a target diagnostic sequence contained in the target molecule, wherein the marker, hook, and vector diagnostic sequence are arranged such that the marker would be removed in the product molecule.

Disclosed are vectors comprising a marker sequence, a hook capable of attaching the vector to a target molecule producing a product molecule, and a vector diagnostic sequence having the same sequence as a target diagnostic sequence contained in the target molecule, wherein the marker, hook, and vector diagnostic sequence are arranged such that the marker would be removed in the product molecule after the vector diagnostic sequence and the target diagnostic sequence recombine or interact.

Disclosed are vectors comprising a marker sequence, a hook capable of attaching the vector to a target molecule, and a vector diagnostic sequence having the same sequence as a target diagnostic sequence contained in the target molecule, wherein the marker, hook, and vector diagnostic sequence are arranged 5′ to 3′ such that the marker would be in between the vector diagnostic sequence and the target diagnostic sequence after the vector and the target molecule are attached via the hook.

Also disclosed are vectors comprising a marker sequence, a first direct repeat sequence, and a first attachment sequence wherein the first attachment sequence can interact to link with a second attachment sequence within a target molecule such that the marker becomes flanked by the first repeat sequence and a second repeat sequence contained within the target molecule and the first direct repeat sequence and second direct repeat sequence can form a tandem repeat sequence.

Disclosed are mixtures comprising a vector comprising a marker sequence, a hook capable of attaching the vector to a target molecule, and a vector diagnostic sequence and a target molecule comprising a target diagnostic sequence, wherein the marker, hook, and vector diagnostic sequence are arranged 5′ to 3′ such that the marker would be in between the vector diagnostic sequence and the target diagnostic sequence after the vector and the target molecule are attached via the hook.

Also disclosed are mixtures comprising a vector and a target molecule wherein the vector comprises a marker sequence, a first direct repeat sequence, and a first attachment sequence and the target molecule comprises a second direct repeat sequence and a second attachment sequence wherein the first attachment sequence and the second attachment sequence can interact to link the vector and the target molecule such that the marker becomes flanked by the first direct repeat sequence and the second direct repeat sequence and the first direct repeat sequence and second direct repeat sequence form a tandem direct repeat sequence.

Also disclosed are vectors, further comprising more than one hook, such as a second hook.

Also disclosed are vectors, wherein the marker sequence encodes a positive selection marker or a negative selection marker.

Also disclosed are vectors, wherein the marker is a protein conferring gentamycin resistance, (G418) hygromycin B (HPH) resistance, nourseothricin (NAT) resistance, blastocidin S (BSR) resistance, or bialaphose (PAT) resistance.

Also disclosed are vectors, wherein the marker sequence encodes a negative selection marker, vectors wherein the marker is URA3, vectors wherein the marker is TRP1, and vectors wherein the marker is CYH2. Also disclosed are vectors, wherein the marker is LYS2 or GAP1. Other negative selection markers can also be used.

Disclosed are vectors, wherein the marker sequence encodes a color marker.

Disclosed are vectors, wherein the color marker is ADE2, vectors wherein the color marker is ADE2-ADE3, and vectors wherein the color marker is SUP11. Also disclosed are vectors, wherein the color marker is ADE2, ADE2-ADE3, MET 25, ASP5, or SUP11.

Disclosed are vectors, wherein the marker confers auxotrophic mutations in a host strain and vectors wherein the auxothrophic mutation marker is LEU2, HIS3, HIS5, THR4, or ARG4.

Also disclosed are vectors wherein the marker sequence encodes a marker protein lethal to a cell.

Disclosed are vectors wherein the hook can recombine with the target molecule.

Also disclosed are vectors, wherein the hook can homologously recombine with the target molecule.

Also disclosed are vectors, wherein the hook can attach to the target molecule through enzymatic manipulation.

Also disclosed are vectors, wherein the enzymatic manipulation includes digestion of the vector.

Also disclosed are vectors, wherein the enzymatic manipulation further includes ligation of the vector.

Also disclosed are vectors, wherein the target diagnostic sequence is endogenous to the target molecule.

Also disclosed are vectors, wherein the target diagnostic sequence is added to the target molecule.

Also disclosed are vectors wherein the vector diagnostic sequence is at least 30, 60, 100, 200, 300, 500, 700, or 1000 bases long.

Also disclosed are vectors wherein the vector diagnostic sequence has at least 75%, 80%, 85%, 90%, or 95% identity to the target diagnostic sequence.

Also disclosed are vectors, wherein after the vector and the target molecule are attached and the distance between the vector diagnostic sequence and target diagnostic sequence after attachment of the vector and the target molecule is less than or equal to 3000 bases, 2000 bases, 1000 bases, 500 bases, 300 bases, or 100 bases.

Also disclosed are vectors, wherein the vector is a TAR vector.

Also disclosed are vectors, wherein the vector further comprises a yeast centromere and a yeast telomere.

Also disclosed are vectors, wherein the vector further comprises an ARS.

1. Vectors

Disclosed are mixtures comprising a vector and a target molecule wherein the vector comprises a marker sequence, a first direct repeat sequence, and a first attachment sequence and the target molecule comprises a second direct repeat sequence and a second attachment sequence wherein the first attachment sequence and the second attachment sequence can interact to link the vector and the target molecule such that the marker becomes flanked by the first direct repeat sequence and the second direct repeat sequence and the first direct repeat sequence and second direct repeat sequence form a tandem direct repeat sequence.

Disclosed are vectors comprising a marker sequence, a first direct repeat sequence, and a first attachment sequence wherein the first attachment sequence can interact to link with a second attachment sequence within a target molecule such that the marker becomes flanked by the first repeat sequence and a second repeat sequence contained within the target molecule and the first direct repeat sequence and second direct repeat sequence can form a tandem repeat sequence.

Disclosed herein are vectors. The disclosed vectors can be used in a variety of techniques, including, for example, gene cloning, library cloning, gene modification techniques, and gene disruption techniques. All of the disclosed vectors have certain common attributes, which aid in the use of the vectors. While each of the parts that may be present in the vector are described herein, it is important to realize that many of these parts function in a coordinated way. Particularly the marker and the diagnostic sequences that are part of the vector work together to provide a powerful way to select molecules that have incorporated the vector. The marker and diagnostic sequences have a relationship such that after the vector has been attached to a target molecule, the marker will be between a diagnostic sequence that was provided by the vector and a diagnostic sequence that was provided by the target molecule. Non-target molecules have almost no chance (the chance is approximately 1 chance in 4²⁴ events) of providing the correct relationship between the marker and the diagnostic sequences, and thus, observable alterations to the molecule formed by the attachment of the vector and the target molecule depend on the correct relationship. Thus, unwanted attachment events can be distinguished from desired attachment events quite efficiently. If the correct attachment event has taken place, the relationship between the marker and the diagnostic sequences will cause the marker to be excised from the molecule comprising the attached vector and target molecule. This excision occurs because of a recombination event that talces place between the diagnostic sequences, wherein the result of the recombination event is the removal of the region that lies between the diagnostic sequences. Each of the parts which may be present in the disclosed vectors are discussed below, however, this is not intended to represent all of the modifications that can be made to the vector that are available.

a) Vector Diagnostic Sequence (DS)

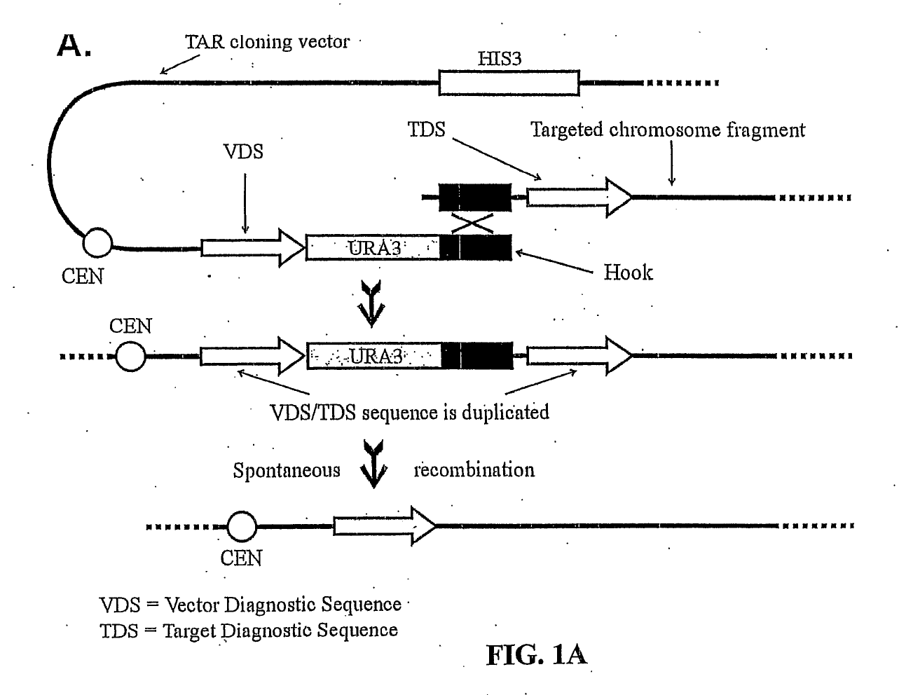

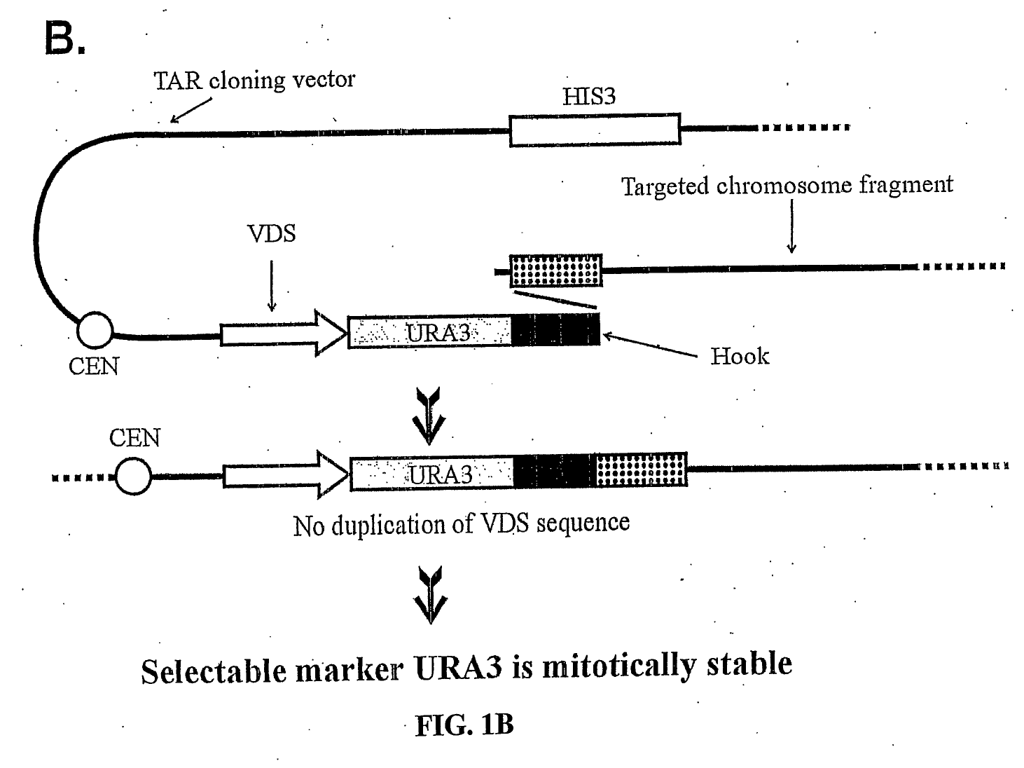

The vector diagnostic sequence is the diagnostic sequence that is part of the vector prior to the attachment of the vector to the target molecule. The vector diagnostic sequence is also related in many ways to the target diagnostic sequence (TDS). The VDS and the TDS typically have a relationship such that when the VDS and the TDS are in proximity to each other events will take place, typically homologous recombination events, that cause the VDS, TDS, and any sequence in between the VDS and the TDS to be reduced to a single VDS/TDS hybrid sequence (see FIG. 1). This VDS/TDS hybrid sequence typically is made of ½ of the original VDS sequence and ½ of the original TDS sequence. This particular relationship between the VDS and the TDS is what allows very specific gene modification and gene disruption events to take place. The relationship between the diagnostic sequences and the marker sequences is what allows for efficient observation of these events, as further described elsewhere herein.

Thus, at one level the VDS must be a sequence that is related in ways described herein to the TDS. The VDS and the TDS are related, and the fuictionality of one is linked to the functionality of the other. This functional relationship is typically the ability to have homologous recombination take place between the VDS and the TDS. Therefore, the disclosed vectors can contain any VDS such that the VDS can recombine with the TDS.

(1) Size

One way to define the VDS is by the size of the VDS region. It is understood that the size of the VDS can affect a number of aspects of the VDS, including but not limited to the ability to recombine, the ability to fit into the vector, and the precision with which the typical recombination event takes place. The lower limit on the size of the VDS will typically be about 30 bases, however, it is understood that this lower limit, for example, is dependent on the sequence of the VDS and the distance the VDS is apart from the TDS, the sequence with which the VDS will interact, or the organism that recombination is occurring in (for example, more efficient recombination takes place in E. coli with regions of about 50 bp). In general, the lower limit on size is controlled by the ability to efficiently hybridize with the TDS, and so as both sequence and effective concentration (i.e. the distance the VDS and the TDS are apart from each other) affect the ability to hybridize, they will also affect the interactions between the VDS and the TDS. Typically the VDS and TDS will be about the same size or exactly the same size.

Certain vectors will have a VDS that is at least about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

Certain vectors will have a VDS that is no greater than about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

(2) Same sequence

Another way to characterize the VDS is by the sequence of the VDS, and by the sequence of the VDS relative to the sequence of the TDS. The VDS must typically have a sequence that is capable of supporting a recombination event between the VDS and the TDS. ((21) and Larionov et al., “Transformation-associated recombination between diverged and homologous DNA repeats is induced by strand breaks.” Yeast 10:930104 (1994) and Mezard, et al., “Recombination between similar but not identical DNA sequences during yeast transformation occurs within short stretches of identity.” Cell 70:656-670 (1992) Shen P., and Huang, Homologous recombination in E. coli: dependency on substrate length and homology. Genetics 112: 441-457 (1986); Watt, V. M., Ingles C. J., Urdea M. S. and Rutter W. J. Homology requirements for recombination in E. coli. Proc. Natl.Acad. Sci USA 82: 4768-4772) (1985) which are herein incorporated by reference at least for material related to homologous recombination and homologous recombination with divergent sequences).

It is understood that recombination events depend on sequence identity between the two regions that are recombining and also, as discussed elsewhere herein, are dependent on for example the size of the region and the distance the two regions are apart. With respect to sequence, however, the VDS can be any sequence that supports a recombination event between the VDS and the TDS. In this regard, the higher the identity between the VDS and the TDS, the greater the efficiency of recombination.

In certain embodiments the VDS has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with the TDS. Typically, the higher the identity is between the VDS and TDS the smaller the VDS and TDS can be for efficient recombination.

It is understood that when determining the identity between a particular VDS and TDS that the identity is calculated between any 30 base cassette within the full length VDS. So, for example, in a certain vector, the VDS sequence may be 100 bases long. The 100 base VDS may have only 70% identity across the full length of the VDS, but there may be a 30 base cassette, within the 100 base VDS that has 100% identity. The VDS as a whole would be said to have 100% identity, based on the 30 base cassette, unless otherwise specifically indicated. Thus, when determining identity between the VDS and the TDS using one of the methods for determining identity discussed elsewhere herein, the identity is calculated by the 30 base cassette within the VDS and TDS with the highest identity. It is understood that identity can also be calculated across the entire length of the VDS and can be used to define the VDS, where indicated.

Another aspect to the issue of sequence, or VDS makeup, arises from the fact that the efficiency of homologous recombination increases as the identity of the terminal most sequence, which is to recombine, increases. Thus, it is more important that the ends of the VDS and the TDS have a certain identity than it is that interior regions of the VDS and TDS have a certain identity. One way of addressing this is to have vectors that have a VDS and TDS which have at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 contiguous bases with 100% identity in at least one of the ends of the VDS and TDS sequence. In still other vectors, at least one of the terminal ends of the VDS and TDS will have between 10 and 50 or between 20 and 50 or between 30 and 50 contiguous bases with 100% identity. In still other vectors, both terminal ends of the VDS and TDS will have at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 contiguous bases with 100% identity. In still other vectors, both terminal ends will have between 10 and 50 or between 20 and 50 or between 30 and 50 contiguous bases with 100% identity.

Typically a VDS and a TDS will have two terminal ends, and typically one of the terminal ends of the VDS will also be a free terminal end which means that the VDS is only flanked by non VDS sequence on either the 3′ or the 5′ end of the VDS. It is understood that because of the relationship between the VDS and the marker there are three possible scenarios: 1) the VDS is immediately juxtaposed to the marker or marker regulatory sequence, 2) all or part of the VDS is made up of all or part of the marker or marker regulatory sequence, or 3) the VDS is not either 1 or 2 which means that there is some type of intervening sequence between the VDS and what is considered the marker or marker regulatory sequence. Unless otherwise indicated, these are the three possible scenarios and then one way of determining where the terminal ends of the VDS and TDS start is by looking at the marker or marker regulatory sequence. In the disclosed vectors, unless otherwise indicated, the terminal end of the VDS is determined by the first base of the VDS that is not considered part of the marker sequence, including any regulatory regions associated with the marker sequence. Of course, when the VDS is part of the marker sequence, or part of any regulatory sequence associated with the marker sequence, then clearly the terminal end of the VDS would include marker or marker regulatory sequence. In such cases, the terminal end of the VDS and the TDS will typically be determined, unless otherwise indicated, by the last base within the TDS that would be considered part of the marker or marker regulatory sequence, were it part of the VDS.

Finally, if there is sequence in between the VDS and the marker or marker regulatory sequence, for example, there could be restriction enzyme sites engineered between the marker sequence and the VDS or any other desired sequence could be intervening. The terminal end of the VDS sequence would occur, unless otherwise indicated, where the first stretch of nucleotides not considered part of the marker or marker regulatory sequence with at least about 5, contiguous bases of 100% identity between the target molecule and the region considered the VDS begins.

It could also be that the terminal ends of the VDS and TDS are determined by the first stretch of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 contiguous bases between the VDS and TDS with 100% identity.

b) Marker Sequences

One part of the vector is the marker or marker sequence. The marker sequence encodes the any type of selectable marker. For example the encoded marker can be a positive selection marker complementing auxotrophic mutations in a host strain (LEU2, HIS3, HIS5, THR4, ARG4) or heterogeneous dominant drug resistance genes such as those conferring resistance to gentamycin (G418) hygromycin B (HPH), nourseothricin (NAT), blastocidin S (BSR) and bialaphos (PAT). The encoded marker can also be a negative selection marker, such URA3, TRP1, CYH2, LYS2, GAP1. The markers can be color markers or other visual markers, such as ADE1, ADE2, ADE2-ADE3, MET 25, ASP5, or SUP11. Since the selection methods disclosed herein rely on the removal of the marker from the molecule formed from the vector and the target molecule attachment, markers where the easy observance of the presence or absence of the marker are contemplated. For example, markers where the presence or absence of the marker effects cell viability or even organism viability are contemplated as well as markers which can be assayed via the presence or absence of an enzymatic reaction.

(1) Marker Regulatory Sequences

The disclosed markers can have any type of regulatory sequence that is appropriate for the expression of the marker. For example, the markers can have regulatory sequence that is constitutive or regulatable. The regulatory sequence can also be cell or organism specific.

The regulatory sequence can be homologous as well as heterologous as in a case of heterogeneous dominant drug resistance genes used as positive selectable marker. Possible regulatory sequences are CUP1 encoding metallothioneine, PHO5 encoding inducible acid phosphatase, GAL1/10 encoding galactoldnase.

(2) Position

As discussed above, the marker of the disclosed vectors has a particular relationship, to the VDS of the vector and also to the TDS contained in the target molecule, once attachment (i.e. recombination or ligation) occurs. This relationship requires that the marker be in between the VDS and the TDS after the target molecule and the vector have been attached, forming a product molecule. If the marker has this arrangement the only other requirement is that the VDS and the TDS be able to recombine as described herein to remove the marker sequence from the product molecule. The marker can be any size or composition and the distance in between the VDS and the TDS in the product molecule may be any size provided the TDS and the VDS are able to function as intended.

In certain product molecules, however, the distance between the VDS and the TDS in the product molecule is less than 500, 600, 700, 800, 900, 1000, 1250, 1500, 1750, 2000, 3000, 3500,4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19,000, 20,000 bases long.

In certain product molecules, however, the distance between the VDS and the TDS in the product molecule is at least 500, 600, 700, 800, 900, 1000, 1250, 1500, 1750, 2000, 3000, 3500, 4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19,000, 20,000 bases long.

It is understood, as discussed herein, that in certain vectors there is overlap between the VDS sequence and the marker and/or regulatory sequence. The marker and regulatory sequence are considered in between the VDS and the TDS if upon recombination of the VDS and TDS the marker sequence is interrupted so that the encoded protein product is atypically functional. For example, if the marker sequence was 2000 bases long, with the bases numbered from 1 to 2000 and the VDS was 1000 bases long, and was defined by nucleotides 1000 to 2000 of the marker, and the TDS contained the same 1000 bases, numbered 1-1000, but contained a single base difference at position 800 which caused a frame shift mutation in the marker so that the protein product was atypically functional. This marker would be considered in between the VDS and the TDS even though part of the marker and/or regulatory sequence was “outside” of the region defined by the positions of the VDS and the TDS in the product molecule. Thus, the key aspect is that the function of the marker gene product will be disrupted by recombination of the VDS and TDS. This can occur when, for example, the relationship between the marker is such that there is VDS sequence, then marker sequence, then TDS sequence. In this relationship, the entire marker region will be excised upon recombination of the VDS and TDS. The functional requirement can also be met, however, by a situation where the relationship between the VDS, marker, and TDS is the following. First the VDS, then intervening sequence, then TDS sequence, then marker sequence, wherein the TDS sequence forms some part of the marker sequence. Now, when recombination takes place, if there is a change in the marker sequence that occurs because of the recombination between the VDS and the TDS, and this change alters the function of the protein encoded by the marker sequence, for example, then the functional requirement has been achieved.

c) Hook

The hook sequences within the vector and within the target molecule are designed to facilitate the attachment of the vector to the target molecule. In certain embodiments, the attachment will become a covalent attachment because of ligation events that occur either within the cell containing the vector and target molecule, or can occur in vitro, on for example, a column containing either the target molecule or the vector. The hook can be involved in homologous recombination events or the hook can attach the vector and the target molecule to each other through, for example, having a region of identity, such that restriction of the vector and target molecule would lead to sticky ends which could then be ligated together.

Attachment could also occur through any other known affinity system. For example, the hooks can be affinity binding pairs that will specifically interact with each other. For example, the hooks can be an avidin:streptavidin or biotin:streptavidin pair or any antigen:antibody pair or antibody:antiantibody pairs, or any of the digoxygenin:[any of the numerous binding molecules] pairs(Kerkhof, Anal Biochem. 205:359-364 (1992)).

Typically the vectors will also have a hook. The hook is a sequence which is designed to facilitate the attachment of the vector to the target sequence. The hook can be a variety of molecules as long as the molecules facilitate an interaction between the vector and the target sequence.

In certain vectors the hook is a nucleic acid sequence which is designed to homologously recombine with a sequence in the target molecule. In that sense, there can be a vector hook and a target molecule hook which have a relationship based on their ability to support homologous recombination. Thus, just as with the VDS and TDS, the size and composition of the hooks can affect the efficiency of homologous recombination between the vector hook and the target hook.

(1) Size

One way to define the hook is by the size of the hook region, when the hook is nucleic acid. It is understood that the size of the hook can affect a number of aspects of the hook, including but not limited to the ability to recombine, the ability to fit into the vector, and the precision with which the typical recombination event takes place. The lower limit on the size of the hook will typically be about 30 bases, however, it is understood that this lower limit, for example, is dependent on the sequence of the hook and the sequence with which the hook will interact. In general, the lower limit on size is controlled by the ability to efficiently hybridize with the target molecule, and so as both sequence and effective concentration (i.e. the distance the vector and target molecule are apart from each other) affect the ability to hybridize, they will also effect the interactions between the hook and the target molecule or the hooks of the target molecule and vector.

Certain vectors will have a hook that is at least about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

Certain vectors will have a hook that is no greater than about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

(2) Same sequence

Another way to characterize the hook is by the composition of the hook and by the composition of the hook relative to the composition of the target molecule. When the hook is nucleic acid, the hook must typically have a sequence that is capable of supporting a homologous recombination event between the hook and the target molecule (Larionov et al., “Transformation-associated recombination between diverged and homologous DNA repeats is induced by strand breaks.” Yeast 10:930104 (1994) and Mezard, et al., “Recombination between similar but not identical DNA sequences during yeast transformation occurs within short stretches of identity.” Cell 70:656-670 (1992) which are herein incorporated by reference at least for material related to homologous recombination and homologous recombination with divergent sequences).

It is understood that recombination events depend on sequence identity between the hook and the target molecule when the hook is nucleic acid, which are recombining and also, as discussed elsewhere herein, are dependent on the size of the hook. With respect to sequence, however, the hook can be any sequence that supports a recombination event between the hook and the target molecule. In this regard, the higher the identity between the hook and the target molecule, the greater the efficiency of recombination.

In certain embodiments the hook has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with the target molecule. Typically, the higher the identity is between the hook and the target molecule the smaller the hook can be for efficient recombination.

It is understood that when determining the identity between a particular hook and target molecule that the identity is calculated between any 30 base cassette within the full-length hook. So, for example, in a certain vector, the hook sequence may be 100 bases long. The 100 base hook may have only 70% identity across the full length of the hook, but there may be a 30 base cassette, within the 100 base hook that has 100% identity. The hook as a whole would be said to have 100% identity, based on the 30 base cassette, unless otherwise specifically indicated. Thus, when determining identity between the hook and the target molecule using one of the methods for determining identity discussed elsewhere herein, the identity is calculated by the 30 base cassette within the hook and the target molecule with the highest identity. It is understood that identity can also be calculated across the entire length of the hook and can be used to define the hook, where indicated.

Another aspect to the issue of sequence or hook composition arises from the fact that the efficiency of homologous recombination increases as the identity of the terminal most sequence, which is to recombine, increases. Thus, it is more important that the ends of the hook and the target molecule have a certain identity than it is that interior regions of the hook and target molecule have a certain identity. One way of addressing this is to have disclosed vectors that have a hook that has at least about 5, 6, 7, 8, 9, 10,11, 12, 13,14, 15, 16,17, 18, 19,20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 contiguous bases with 100% identity to at least part of the target molecule or end of the target molecule. In still other vectors, at least one of the terminal ends of the hook and target molecule will have between 10 and 50 or between 20 and 50 or between 30 and 50 contiguous bases with 100% identity. In still other vectors, both terminal ends will have at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46,47, 48, 49, 50 contiguous bases with 100% identity. In still other vectors, both of the terminal ends of the hook and target molecule complementary regions will have between 10 and 50 or between 20 and 50 or between 30 and 50 contiguous bases with 100% identity.

Typically a hook will have two terminal ends, but only one free terminal end. It is understood that because of the relationship between the hook and the marker there are three possible scenarios: 1) the hook is immediately juxtaposed to the marker or marker regulatory sequence, 2) all or part of the hook is made up of all or part of the marker or marker regulatory sequence, or 3) the hook is not either 1 or 2 which means that there is some type of intervening sequence between the hook and what is considered the marker or marker regulatory sequence. Unless otherwise indicated, these are the three possible scenarios and then one way of determining the terminal ends of the hook is by looking at the marker or marker regulatory sequence. In the disclosed vectors, unless otherwise indicated, the terminal end of the vectors is determined by the first base of the hook that is not considered part of the marker sequence, including any regulatory regions associated with the marker sequence. Of course, when the hook is part of the marker sequence, or part of any regulatory sequence associated with the marker sequence, then clearly the terminal end of the hook would include marker or marker regulatory sequence. In such cases, the terminal end of the hook will typically be determined, unless otherwise indicated, by the last base within the target molecule that would be considered part of the marker or marker regulatory sequence, were it part of the vector.

Finally, if there is sequence in between the hook and the marker or marker regulatory sequence, for example, there could be restriction enzyme sites engineered between the marker sequence and the hook or any other desired sequence could be intervening, the terminal end of the hook sequence would occur, unless otherwise indicated, where the first stretch of nucleotides not considered part of the marker or marker regulatory sequence with at least about 5, contiguous bases of 100% identity between the target molecule and the region considered the hook begins.

Just as one of the hooks is typically determined by the position of the hook relative to the marker, the end of the hook farthest away from the marker sequence and/or marker regulatory sequence is typically determined by the end of the sequence having homology with the target molecule. For example, the end of the sequence having homology with the target molecule could be determined by the last sequence of the hook having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous bases of interaction with the target molecule. In this situation, then the last base would be considered the last contiguous base, farthest from the marker and/or marker regulatory sequence.

It could also be that the terminal ends of the hook are determined by the first stretch of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 contiguous bases between the hook and target molecule.

It is understood that there will be a region in the target molecule that is also sequence which could be referred to as a hook, because it is sequence that will be involved in the recombination event, for example, with the vector. The same types of parameters that can define the hook in the vector can also define a hook region in the target molecule, except for the discussion about the terminal ends. This is because the hook has no relationship to the marker sequence until after the vector and the target molecule have become attached. Therefore is understood that the ends of a hook region within the target molecule will be solely defined by the ends of the hook within the vector that will be mixed with the target molecule.

(3) Specific Types of Hook Sequences

(i) Repeat Regions

For example, the sequence on the vector which can recombine with a region of a nucleic acid within the population of nucleic acids can comprise a repeat sequence, such as an Alu repeat. (See, Watson, et al., “Recombinant DNA” 2nd ed, Dist. by W. H. Freeman and Co., New York, 1992). Therefore the sequence of the vector which can recombine with a region of the nucleic acid within the mixed population of nucleic acids recombines with a repeat sequence on a nucleic acid within the population of nucleic acids.

Where the sequence on the vector which can recombine with a region of a nucleic acid within a population of nucleic acids comprises a short interspersed element (SINES), such as an Alu repeat, recombination between the sequence on the vector and a similar sequence on the nucleic acid may be at any one of a plurality of sites on the nucleic acid. For example, a population of nucleic acids from a particular organism, such as a human, may contain multiple Alu repeats and recombination between a vector sequence comprising an Alu repeat and an Alu repeat or Alu-like repeat sequence on a nucleic acid within the population of nucleic acids may occur at various sites on the nucleic acid.

(4) Number of Hook Sequences

The vectors can have at least one hook. However, the vectors can have more than one hook, such as two hooks. The hooks can be different sequences and designed to interact with different parts of a target molecule. The hooks can also be in any orientation relative to each other. Typically if the vector is a vector designed to form a linear molecule with the target molecule, there will be only one hook, however, there can be more than one hook in this type of vector. Typically in a vector designed to form a circular molecule with the target molecule, or a portion of the target molecule, this type of vector will typically have at least two hooks.

In certain embodiments the hook may actually be all or part of the VDS sequence as long as the basic requirement that loss of a marker sequence occurs because of the attachment of the vector to the target molecule.

d) Target Diagnostic Sequence (TDS)

The target diagnostic sequence, which is contained in the target molecule prior to attachment to the vector, is the counterpart sequence to the VDS. The TDS is also related in many ways to the VDS. Those aspects of the VDS, not specifically discussed for the TDS can also apply to the TDS unless specifically indicated otherwise.

The VDS and the TDS are related, and the functionality of one is linked to the functionality of the other. This functional relationship is typically the ability to have homologous recombination take place between the VDS and the TDS. Therefore, the disclosed vectors can include any VDS such that the VDS can recombine with the TDS.

(1) Size

One way to define the TDS is by the size of the TDS region. It is understood that the size of the TDS can affect a number of aspects of the TDS, including but not limited to the ability to recombine, and the precision with which the typical recombination event takes place. The lower limit on the size of the TDS will typically be about 30 bases, however, it is understood that this lower limit, for example, is dependent on the sequence of the TDS and the distance the TDS is apart from the VDS, the sequence with which the TDS will recombine. In general, the lower limit on the size required for recombination is controlled by the ability to efficiently hybridize with the VDS. As both sequence and effective concentration (i.e. the distance the TDS and the VDS are apart from each other) affect the ability to hybridize, they will also affect the recombinations between the TDS and the VDS.

Certain vectors will have a TDS that is at least about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

Certain vectors will have a TDS that is no greater than about, 20, 25, 30, 35, 40, 45, 50, 55, 60, 65, 70, 75, 80, 85, 90, 95, 100, 125, 150, 175, 200, 225, 250, 300, 325, 350, 375, 400, 425, 450, 475, 500, 600, 700, 800, 900, 1000, 1250, 1500, 1750, 2000, 3000, 4000, or 5000 bases long.

(2) Same Sequence

Another way to characterize the TDS is by the composition of the TDS, and by the composition of the TDS relative to the composition of the VDS. The TDS must typically have a sequence that is capable of supporting a homologous recombination event between the TDS and the VDS. It is understood that recombination events depend on sequence identity between the two regions, which are recombining and also, as discussed elsewhere herein, are dependent on for example the size of the region and the distance the two regions are apart. With respect to sequence, however, the TDS can be any sequence that supports a recombination event between the TDS and the VDS. In this regard, the higher the identity between the TDS and the VDS, the greater the efficiency of recombination.

In certain embodiments the TDS has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with the VDS.

Typically, the higher the identity is between the TDS and the VDS the smaller the TDS can be for efficient recombination.

It is understood that when determining the identity between a particular TDS and VDS that the identity is calculated between any 30 base cassette within the fill length TDS. So, for example, in a certain vector, the TDS sequence may be 100 bases long. The 100 base TDS may have only 70% identity across the full length of the TDS, but there may be a 30 base cassette, within the 100 base TDS that has 100% identity. The TDS as a whole would be said to have 100% identity, based on the 30 base cassette, unless otherwise specifically indicated. Thus, when determining identity between the TDS and the VDS using one of the methods for determining identity discussed elsewhere herein, the identity is calculated by the 30 base cassette within the TDS and VDS with the highest identity. It is understood that identity can also be calculated across the entire length of the TDS and can be used to define the TDS, where indicated.

The terminal ends of the TDS are typically determined by the sequence that interacts with the defined VDS. As discussed above, the VDS is typically defined by the relationship between the VDS and the marker and/or marker regulatory sequence. Likewise the TDS is thus, typically defined by the VDS, and typically would be considered the sequence that hybridizes, and thus recombines, with the VDS. Understanding that not every base within the TDS must hybridize with the VDS one way of determining the ends of the TDS is by looking at the number of contiguous bases of hybridization present in the TDS with the VDS. For example, the terminal ends of the TDS can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous bases of hybridization with the VDS. In still other vectors, at least one of the terminal ends of the TDS and VDS will have between 10 and 50 or between 20 and 50 or between 30 and 50 contiguous bases with 100% identity.

It is understood that the there could be for example, 30 contiguous bases of hybridization within the TDS, but only 15 contiguous bases of hybridization within the VDS when for example, there was a single base mismatch within the VDS. In other words, the contiguous nature of the VDS and the TDS are independent with respect to size.

e) Target Molecule

The target molecule can be any molecule with which it is desired that the disclosed VDS vectors interact. The target molecule typically will have the parts, such as a hook and a TDS endogenously contained within the target molecule. In this type of situation the VDS vector was designed to meet the requirements of the target molecule. In other situations the target molecule will itself be engineered to have for example, a hook and a TDS.

There are many ways in which the target molecule may be obtained or chosen. If the target molecule is to be engineered, for example, on a library of molecules, the library molecules can all be modified, for example by the addition of an adaptor molecule which typically will contain a hook sequence and/or a TDS.

The target molecule can be within a population of nucleic acids, or could in fact be a population of nucleic acids, all of which are related in some way, for example, by a related hook region. When the target molecule is a population of molecules each member of the population is also considered a target molecule. The target molecule can also be a single molecule contained within the cell, which is not native to the cell, as well as a nucleic acid or region of nucleic acid contained within a cell that is native to the cell. For example, the target molecule could be a plasmid molecule transfected in the cell or it could be a region of a gene contained on one of the chromosomes within the cell.

f) Other Components which may be Part of the Disclosed Vectors

Any other part of a vector can typically be incorporated into the disclosed VDS vectors. For example, promoters, enhancers, and cloning sites can all be added to the disclosed VDS vectors. The vectors can also be used with recombination systems that promote recombination, such as the RecA system.

2. Other Nucleic Acids

a) Primers and Probes

Disclosed are compositions including primers and probes, which are capable of interacting with the VDS vectors, or the molecules formed from interaction between the VDS vector and the target molecule, as disclosed herein. For the purpose of the primers and probes, the VDS vector primer or probe will represent all of the possible targets related to the VDS vector, such as the molecule formed from the interaction of the VDS vector with the target molecule. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the VDS vectors or region of the VDS vectors or they hybridize with the complement of the VDS vectors or complement of a region of the vectors.

Likewise primers and probes that hybridize with the marker sequence, attachment region where capable, target molecule, first repeat region, second repeat region, first attachment region, and second attachment region or the complements of each of these.

The size of the primers for interaction with the VDS vectors in certain embodiments can be any size that supports the desired enzymatic manipulation of the primer, such as DNA amplification. A typical VDS vector primer would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a VDS vector primer can be less than or equal to 6, 7, 8,9, 10, 11, 12 13, 14, 15, 16, 17, 18, 19,20,21, 22, 23, 24, 25, 26,27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the VDS vector typically will be used to produce an amplified DNA product that contains the VDS vector.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

3. Compositions Related to Nucleic Acids

Disclosed are cells comprising any of the disclosed nucleic acids. The cells can be any type of cell capable of being transformed or transfected with the disclosed nucleic acids in a way that allows the marker to be removed. The cells can be for example, prokaryotic cells or eukaryotic cells. The cells can be, for example, yeast cells, such as s. cerevisiae, or the cells can be for example mammalian cells, such as mouse or rat or rabbit or ovine or porcine or bovine or primate, such as monkey, orangutan, chimpanzee, ape, or human.

Disclosed are animals comprising any of the nucleic acids or peptides or cells disclosed herein. The animals can be any animal including mammals, such as mouse or rat or rabbit or ovine or porcine or bovine or primate, such as monkey, orangutan, chimpanzee, ape, or human.

4. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit comprising a vector comprising a VDS and a marker as well as buffers needed for the vectors manipulation.

5. Chips and Micro Arrays

Disclosed are chips and microarrays of any nature that can interact with any part of the disclosed vectors or product molecules made from the attachment of the vectors with the target molecule. Any method of identifying the presence of the vectors and the product molecules is disclosed herein. The identification can take place at any step in the disclosed processes. For example, the identification can take place after vector construction or after vector addition to a cell or after isolation of a product molecule from a cell.

Disclosed are chips wherein at least one address on the chip is the sequence or sequence complement to the disclosed vectors or product molecules.

Disclosed are chips wherein at least one address on the chip is a sequence related to the disclosed vectors or product molecules.

Also disclosed are chips wherein at least one address is a variant of the disclosed vectors or product molecules. Methods of using the chips and microarrays are also disclosed.

### D. Methods of Making the Compositions

The compositions disclosed herein and the compositions necessary to perform the disclosed methods can be made using any method known to those of skill in the art for that particular reagent or compound unless otherwise specifically noted.

1. Vectors

The vectors can be made using any process known in the art. For example, the vectors can be made using standard recombinant biotechnology methods disclosed in Sambrook et al. or Ausebel et al., Molecular Cloning: A Laboratory Manual, 2nd Edition (Cold Spring Harbor Laboratory Press, Cold Spring Harbor, N.Y., 1989).

2. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### E. EXAMPLES

Unless indicated otherwise, parts are parts by weight, temperature is in ° C. or is at ambient temperature, and pressure is at or near atmospheric.

### 1. Example 1

### Materials and Methods

a) Yeast Strains, Transformation and Selection of Gene-Positive Clones.

The highly transformable Saccharomzyces cerevisiae strain VL6-48N (MATa, his3-Δ200, trp1-Δ1, ura3-Δ1, lys2, ade2-101, met14, cir^{o}), which has deletions of HIS3 and URA3, was used for transformations. The strain was generated from VL6-48 (11) by substitution of the ura3-52 gene with a KanMX cassette (17). Spheroplasts were generated as described previously (18). Agarose plugs (100 μl) containing approximately 5 μg of high molecular weight DNA were prepared with DNA from normal human fibroblasts MRC-5 (ATCC) or from liver cells of the Tg.AC mouse (12). Linearized TAR cloning vector (1 μg) was added to the DNA-containing plugs, treated with agarase, and mixed with yeast spheroplasts. Transformants were selected on synthetic complete medium plates lacking histidine. To identify gene-positive clones, His⁺ Ura⁺ primary transformants were replica plated on synthetic histidine minus plates containing 5-fluoroorotate (5-FO) to select clones with the unstable URA3 marker (19).

To estimate density of an ARS sequence(s) in mouse and human genomic DNA fragments, DNAs were isolated from randomly selected clones of large size inserts BAC libraries constructed with the pTARBAC1 vector containing a yeast selectable marker (HIS3) and centromere (CEN6) (20). Size of inserts in the libraries varies from 130 to 200 kb. Typically between 200 and 1,000 transformants were obtained during standard spheroplast transformation with 10 ng of BAC DNA if the BAC contained an ARS-like sequence.

b) Construction of TAR Cloning Vectors.

A new TAR vector, pVC604-HP, containing the URA3 negative-selectable marker, two targeting sequences (hooks) and a VDS was constructed using the basic TAR cloning vector pVC604 (HIS3-CEN6) (18). Two hooks, a 148 bp of the 3′ sequence of the human HPRT gene and a 189 bp of the Alu BLUR13 sequence, were PCR amplified from the pVC-BP1 vector (HPRT-CEN6-HIS3-Alu) that was previously used for TAR cloning of the human HPRT gene (11). A 148 bp HPRT hook [positions 53,695-53,842 in genomic sequence (accession number M26434)] was cloned as a SalI-EcoRI fragment and a 189 bp Alu hook was cloned as a ApaI-XhoI fragment into the pVC604 polylinker. A 1,001 bp HPRTVDS flanking the gene-specific hook was PCR amplified from the HPRTYAC (11) and cloned in front of the unique hook as a BamHI-XbaI fragment. The VDS directly lies downstream of the hook sequence in the HPRT genomic sequence. This sequence [positions 52,694-53,694 in genonic sequence (accession number M26434)] is not entire unique. A 379 bp of the sequence corresponds to a LINE1 transposable element. The URA3 gene was PCR amplified from pRS306 as ˜1.1 kb EcoRI-BamHI fragment and cloned between the unique hook and the VDS. A schematic representation of the pVC604-HP vector is shown in FIG. 1.

TAR vector pVC604-Tg used for cloning of the mouse Tg.AC transgene was constructed based on the previously described vector pCV604-B1/SV (12). A 160 bp transgene-specific hook and a 130 bp B1 repeat were re-cloned into the basic TAR cloning vector pVC604 as BamHI-XbaI and SacI-SacII fragments, correspondingly. Different size VDSs (211 bp, 500 bp and 1,000 bp of the v-Ha-ras gene sequence) those lie distal to a targeting hook sequence in the transgene were PCR amplified from the transgene-containing YAC (16) and cloned in front of the hook as XhoI-EcoRI fragments. The URA3 gene was PCR amplified as a 1.1 kb EcoRI-BamHI fragment and cloned between the unique hook and the VDS. The HPRT TAR cloning vector was cut with SalI; transgene vectors were cut with NotI (these sites are located between the hooks) before transformation to yield linear molecules bounded by gene-specific hook on one end and a common repeated DNA element (i.e., Alu, B1) as a hook on the other end.

c) PCR Analysis.

Two pairs of primers were used to characterize HPRTYACs by PCR. IN1R/IN1L amplify a 516 bp sequence of intron 1 and 46L/47R amplify a 575 bp sequence of exon 2 along with flanking introns (11), The results of this PCR reaction indicate which clones formed by recombination between the TAR vector and the 3′ region of the genomic HPRT. The HPRTYAC clones were further characterized using 9 pairs of PCR primers that amplify HPRT exons 1-9 (11). A pair of primers, ZG-F and ZG-R, specific to a zeta-globin promoter region was used for PCR screening of transformants for presence of the Tg.AC transgene sequence (12). These primers generate a 419 bp PCR product that is diagnostic for recombination between TAR vector and genomic Tg.AC transgene sequences, Yeast genomic DNA was isolated from transformants and PCR amplified as described previously (11, 16).

d) Characterization of YAC Clones.

Chromosomal size DNA from yeast transformants was separated by Transverse Alternating Field Electrophoresis (TAFE), blotted and hybridized with a gene specific probe as described previously (11, 16). The size of circular YACs was estimated by digesting agarose DNA plugs with NotI and TAFE gel analysis. Rescue of YAC ends for sequencing was done using standard protocols.

### 2. Example 2

### Results

a) TAR Cloning Strategy Using Negative and Positive Selection

A schematic diagram of a novel TAR cloning strategy that uses positive and negative selection is shown in FIG. 1. Genomic DNA and linearized TAR cloning vector are combined with yeast spheroplasts. The TAR cloning vector contains a yeast centromere (CEN6), a positive-selectable marker (HIS3), a negative-selectable marker (URA3) and two targeting hooks. (Only one hook is shown in FIG. 1; another hook can be either a unique gene-specific sequence or a common repeat). The sequence of the hook used is shown in SEQ ID NO:1. It is a 148 base sequence that can recombine with nucleotides 53695-53842 of the HPRT sequence found in GenBank Accession No. M26434. The TAR vector also carries an additional gene-specific DNA sequence called a VDS that is immediately adjacent but distal to the gene-specific targeting hook sequence in the chromosomal DNA. In the TAR vector, this VDS is proximal to the gene-specific hook, and separated from it by URA3, the negative-selectable marker. This allows for negative selection against the URA3 gene, which is destabilized in clones with the desired gene, because it is flanked by direct repeats in the YAC clone. The sequence of the repeat, VPS, used can be found in SEQ ID NO:2. This sequence can recombine with nucleotides 52694-53694; of the HPRT sequence found in GenBank Accession No. M26434.

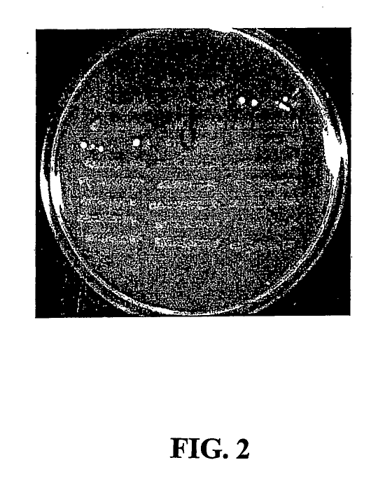

FIG. 2 shows the results of two different recombination events involving chromosomal DNA and the TAR cloning vector (only the gene-specific hook is shown). In FIG. 1A, the TAR vector recombines with the gene-specific targeting hook by homologous recombination; the YAC product then transiently carries (from proximal to distal) the VDS, URA3, the gene-specific targeting hook, and the target molecule diagnostic sequence (TDS). Direct repeats are extremely unstable in yeast, so that there is a high probability for loss of URA3 by spontaneous mitotic recombination involving the direct repeat copies of the VDS and TDS. After this loop-out recombination event, the YAC carries (from proximal to distal) one copy of a hybrid VDS and TDS, followed by distal chromosomal DNA from the gene of interest (FIG. 1A, bottom). If the negative-selectable marker is URA3, positive YAC clones carrying the desired gene can be selected by growth in the presence of 5-fluoroorotate. Other negative-selectable markers can be used in place of URA3.

FIG. 1B shows the results of non homologous recombination or non-homologous end-joining between a chromosomal DNA fragment and the TAR cloning vector. In this case, the VDS and TDS are not together, no direct repeat forms and the URA3 marker is mitotically stable (FIG. 1B).

b) Highly Efficient Cloning of a Multicopy Mouse Transgene

The TAR cloning strategy described above was used to isolate the ν-Ha-rasTg.AC transgene cassette from mouse DNA. The Tg.AC transgenic mouse carries approximately 40 copies of the transgene integrated into a unique site on chromosome 11 (ref. 16 and references therein). Each transgene includes the ν-Ha-ras gene and a simian virus 40 (SV40) polyadenylation signal and is under control of a zeta-globin promoter. The transgene and flanking genomic sequences were recently isolated by radial TAR cloning using a vector carrying 160 bp of SV40 as a transgene-specific targeting hook and a common mouse repeat B1 as a second hook (12). Transgene-positive clones were obtained at a frequency of approximately 2%, at least in part because of the high copy number of the target. In this experiment, the TAR cloning vector was modified to include a VDS, namely 1,000 bp of the ν-Ha-ras gene sequence that lies distal to the transgene-specific targeting hook in the transgene on chromosome 11. In the TAR cloning vector, the VDS and URA3 negative selectable marker were arranged in the configuration described above (see FIG. 1).

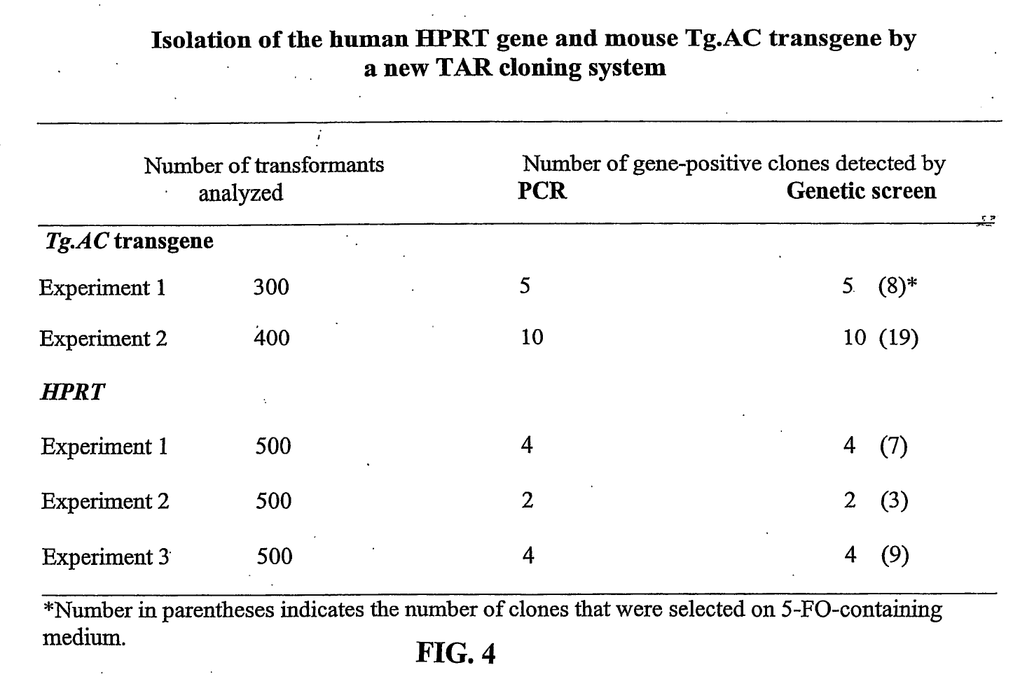

Cloning experiments with the modified vector demonstrated that the yield of transgene-positive clones is also approximately 2% (FIG. 4.). Fifteen positive YAC clones were identified by screening 700 His⁺ transformants by PCR with primers for the zeta globin promoter of the transgene. CHEF analysis showed that the YACs were circular and ranged in size from 50 kb to larger than 200 kb (data not shown), which is typical of clones isolated by radial TAR cloning (12, 16). Stability of the URA3 marker in the transgene-positive transformants was determined by replica plating in the presence of 5-FO. All 15 transformants exhibit a papillae growth on 5-FO plates, indicating that they have lost the URA3 marker by “looping out.” This recombinational loss of URA3 occurs because the VDS is located in the vector and the TDS is located in the insert of the recombinant YAC, forming a direct repeat that flanks the URA3 gene (FIG. 1A). Stability of the URA3 marker was also characterized in the 700 His⁺ transformants used in the above analysis. Twenty seven of 700 colonies exhibited a papillae growth in the presence of 5-FO, and 15 of these 27 carried the v-Ha-ras transgene, as determined by PCR. Between five and hundred Ura7⁻ “pop-out” events were observed on replica of gene-positive colonies (FIG. 2). In contrast, most false-positive transformants (9 from 12) produces 1-2 Ura-colonies when replica plated on 5-FO medium. These Ura7⁻ cells resulted from rare mutations in URA3.

This result indicates that this novel TAR cloning method efficiently identifies targeted recombinants carrying the gene of interest by using negative selection against URA3. Thus, when used to isolate a multicopy gene, this TAR cloning strategy provides a highly efficient method to genetically select for positive clones.

c) Highly Efficient Cloning of a Single Copy Human Gene

TAR cloning with negative selection can also be used to isolate a single copy gene from the human genome. The human HPRT gene was recently isolated by radial TAR cloning (11). The TAR cloning vector used to isolate human HPRT carried a 381 bp 3′ HPRT-specific targeting hook and a 189 bp Alu hook. For this radial TAR cloning experiment, approximately 0.6% of the clones were HPRT-positive (11, 12 and unpublished data).

A similar experiment was performed with a modified TAR cloning vector which included a shorter HPRT-specific hook (148 bp), an 1,001 bp HPRT gene fragment (the VDS) adjacent to the specific hook sequence in chromosomal DNA. As above, the modified vector also carried the negative-selectable marker URA3 (FIG. 1). 1,500 random His+transformants from 3 experiments were selected and replica plated on medium with 5-FO to identify clones with the mitotically unstable URA3. Nineteen colonies exhibited a papillae growth in the presence of 5-FO and 10 of these 19 also carry all the HPRT exon sequences based on a PCR assay (FIG. 4.). CHEF analysis indicated that the HPRT-positive YACs were circular and ranged in size from 70 to 300 kb (data not shown). When the same,1500 transformants were analyzed by PCR for presence of HPRT sequences, ten clones were found those include all exon sequences of HPRT. These results indicate that this novel TAR cloning system is highly efficient, highly selective and sufficiently sensitive to isolate a single copy gene from a large and complex mammalian genome.

d) Molecular Mechanism of Non-Targeted Recombination during TAR Cloning

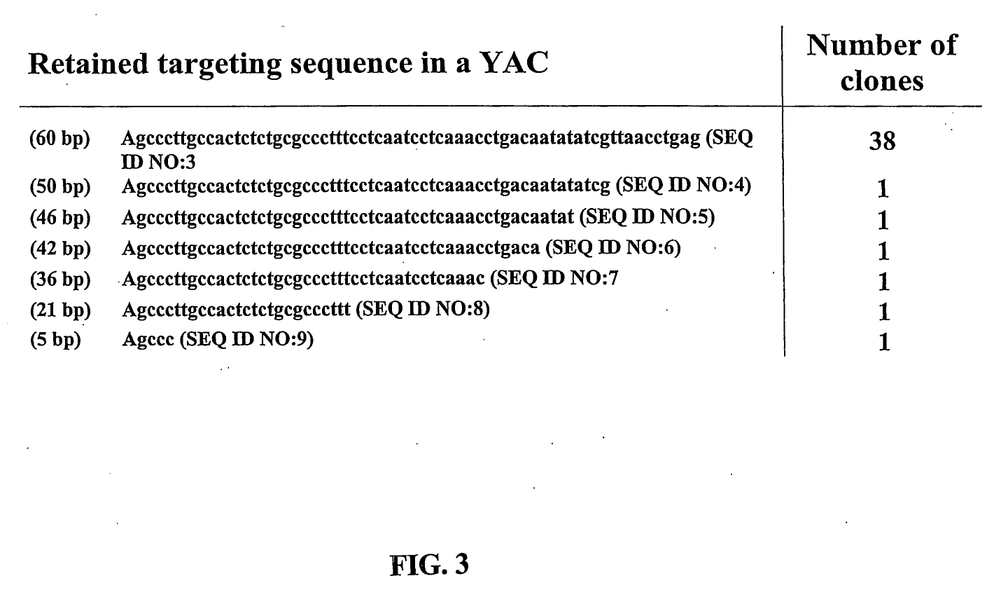

Without negative selection TAR cloning produces recombinant YACs that in most cases carry random genomic fragments instead of the desired gene. These background clones may form by non-homologous end-joining between the vector ends and chromosomal DNA or by homologous recombination between similar but not identical sequences in the vector and chromosomal DNA. To understand these mechanisms, background YAC clones were characterized from a radial cloning experiment that used a TAR vector with a 60 bp HPRT-specific targeting hook and a 189 bp hook from the 5′ end of Alu. This vector had a similar cloning efficiency as the TAR cloning vector containing a bigger size HPRT targeting hook (11, 12) and made it easier to obtain DNA sequence from the insert of the background YACs. The terminal sequences of YAC inserts were rescued as plasmids in E. coli and sequenced using T3 or T7 primers. All of the YAC inserts had an entire Alu sequence at one end, as predicted from homologous recombination that occurred between the TAR vector and a chromosomal Alu sequence. The YAC sequences adjacent to the gene-specific targeting hook are summarized in FIG. 3. Majority of the clones (38 among 44 YAC analyzed) had the entire hook sequence. The sequence of 25 YAC inserts were found in the draft human genome sequence. These sequences had no detectable homology to the HPRT-specific targeting hook in the targeted chromosomal region. This result strongly suggests that the end of the linear TAR vector was ligated to a random chromosomal fragment by an end joining reaction. A minor fraction of the clones (6/44) contained a partial HPRT-specific targeting hook that was 6 to 50 bp long. End sequencing of these clones also showed no homology between the cloned genomic fragments and the HPRT specific targeting hook. These clones could have formed by a combination of nuclease degradation and non-homologous end-joining. In summary, these data indicate that non-homologous end-joining is the main mechanism by which background clones are generated during TAR cloning in yeast.

The technique can be used even when the amount of genomic DNA is a limiting factor (i.e., for clinical studies or to isolate a gene from an obligate parasite that cannot be cultivated outside of its host).

### F. References

1. Burke, D. T., Carle, G. F. and Olson, M. V. (1987) Science 236, 806-812.

2. Shizuya, H., Birren, B., Kim, U.-J., Mancino, V., Slepax, T., Tachiiri, Y. and Simon, M. (1992) Proc. Nat. Acad. Sci. USA 89, 8794-8797.

3. Ketner, G.,Spencer, F., Tugendreich, S., Connelly, C. & Hieter, P. (1994) Proc. Nat. Acad. Sci. USA 91, 6186-6190.

4. Larionov, V., Kouprina, N., Graves, J., Chen, X.-N., Korenberg, J. R. and Resnick, M. A. (1996) Proc. Natl. Acad. Sci. USA 93, 491-496.

5. Ma, H., Kunes, S., Schatz, P. J., and Botstein, D. (1987) Gene 58, 201-216.

6. Erickson, J, R, and Johnston, M. (1993) Genetics 134, 151-157.

7. Pompon, D. and Nicolas, A. (1989) Gene 83, 15-24.

8. Bradshaw, S. M., Bollelcens, J. A. and Ruddle, F. H. (1995) Nucl. Acids Res. 23, 4850-4856.

9. Stinchomb, D. T., Thomas, M., Kelly, I., Selker E. and Davis, R. W. (1980) Proc. Natl. Acad. Sci. USA 77, 4559-4563.

10. Larionov, V., Kouprina, N., Solomon, G, Barrett, J. C. and Resnick, M. A. (199 c17) Proc. Nat. Acad. Sci. USA 94, 7384-7387.

11. Kouprina, N., Annab, L., Graves, J., Afshari, C., Barrett, J. C., Resnick, M. A., and Larionov V. (1998) Proc. Nat. Acad. Sci. USA 95, 4469-4474.

12. Noskov, V., Koriabine, M., Solomon, G., Randolph, M., Barrett, J. C., Leem, S.-H., Stubbs, L., Kouprina, N., and Larionov, V. (2001) Nucl. Acids Res. 29, E62.

13. Cancilla, M., Tainton, K., Barry, A., Larionov, V., Kouprina, N., Resnick, M., Du Sart, D., and Choo, A. (1998) Genomics 47, 399-404.

14. Annab, L., Kouprina, N., Solomon, G., Cable, L., Hill D., Barrett, J. C., Larionov, V., and Afshari, C. (2000) Gene 250, 201-208.

15. Kim, J., Noskov, V. N., Lu, X., Bergmann, A., Ren, X., Warth, T., Richardson, P., Kouprina, N. and Stubbs, L. (2000) Genome Res. 10, 1138-1147.

16. Humble, M., Kouprina, N., Noskov, V., Graves, J., Garner, E., Tennant, R., Resnick, M. A., Larionov, V., and Cannon, R. E. (2000) Genomics 70,292-299.

17. Wach, A, Brachat, A., Pohlmann, R. and Philippsen, P. (1994) Yeast 10, 1793-17808.

18. Kouprina, N. and Larionov, V. (1999) CurrentProtocols in Human Genetics 1, 5.17.1-5.17.21.

19. Boeke, J. D., Trueheart, J., Natsoulis, G. and Fink, G. R. (1987) Methods Enzymol. 154, 164-175.

20. Osoegawa, K., Mammoser, A. G., Wu, C., Frengen, E., Zeng, C, Catanese, J. J. and de Jong, P. (23001) Genome Res. 11, 483-496.

21. Myung, K, Datta A, Chen, C and Kolodner, R. D. (2001) Nat. Genet. 27, 113-116.

22. Theis, J. F., and Newlon, C. S. (1997) Proc. Natl. Acad. Sci. USA 94, 10786-1079.

23. Lewis, L, K. and Resnick M. A. (2000) Mutat. Res. 451, 71-89.

What is claimed is:

1. A vector capable of interacting with a target molecule to produce a product comprising a marker sequence and a sequence which will recombine with a target molecule so that the marker sequence is removed from the product.

2. A vector comprising a marker sequence, a hook capable of attaching the vector to a target molecule, and a vector diagnostic sequence having the same sequence as a target diagnostic sequence contained in the target molecule, wherein the marker, hook, and vector diagnostic sequence are arranged 5′ to 3′ such that the marker would be in between the vector diagnostic sequence and the target diagnostic sequence after the vector and the target molecule are attached via the hook.

3-100 cancelled.

101. The vector of claim 1, further comprising a second hook.

102. The vector of claim 1, wherein the marker sequence encodes a positive selection marker.

103. The vector of claim 1, wherein the marker is protein conferring gentamycin resistance, (G418) hygromycin B (HPH), nourseothricin (NAT), blastocidin S (BSR), or bialaphos (PAT).

104. The vector of claim 1 wherein the marker sequence encodes a negative selection marker.

105. The vector of claim 104, wherein the marker is URA3.

106. The vector of claim 104, wherein the marker is TRP1 .

107. The vector of claim 104, wherein the marker is CYH2, LYS2, or GAP1.

108. The vector of claim 104 wherein the marker confers auxotrophic mutations in a host strain.

109. The vector of claim 108 wherein the marker is LEU2, HIS3, HIS5,THR4, or ARG4.

110. The vector of claim 1 wherein the marker sequence encodes a color marker.

111. The vector of claim 110 wherein the color marker is ADE2.

112. The vector of claim 110, wherein the color marker is ADE2, ADE2-ADE3, MET 25, ASP5, or SUP11.

113. The vector of claim 110 wherein the color marker is SUP11.

114. The vector of claim 1 wherein the marker sequence encodes a marker protein lethal to a cell.

115. The vector of claim 1 wherein the hook can recombine with the target molecule.

116. The vector of claim 1, wherein the hook can homologously recombine with the target molecule.

117. The vector of claim 1, wherein the hook can attach to the target molecule through enzymatic manipulation.

118. The vector of claim 117, wherein the enzymatic manipulation includes digestion of the vector.

119. The vector of claim 116, wherein the enzymatic manipulation further includes ligation of the vector.

120. The vector of claim 1, wherein the target diagnostic sequence is endogenous to the target molecule.

121. The vector of claim 1, wherein the target diagnostic sequence is added to the target molecule.

122. The vector of claim 1, wherein the vector diagnostic sequence is at least 30 bases long.

123. The vector of claim 1 wherein the vector diagnostic sequence is at least 60 bases long.

124. The vector of claim 1 wherein the vector diagnostic sequence is at least 100 bases long.

125. The vector of claim 1 wherein the vector diagnostic sequence is at least 200 bases long.

126. The vector of claim 1 wherein the vector diagnostic sequence is at least 300 bases long.

127. The vector of claim 1 wherein the vector diagnostic sequence is at least 500 bases long.

128. The vector of claim 1 wherein the vector diagnostic sequence is at least 700 bases long.

129. The vector of claim 1 wherein the vector diagnostic sequence is at least 1000 bases long.

130. The vector of claim 1 wherein the vector diagnostic sequence has at least 75% identity to the target diagnostic sequence.

131. The vector of claim 1 wherein the vector diagnostic sequence has at least 80% identity to the target diagnostic sequence.

132. The vector of claim 1 wherein the vector diagnostic sequence has at least 85% identity to the target diagnostic sequence.

133. The vector of claim 1 wherein the vector diagnostic sequence has at least 90% identity to the target diagnostic sequence.

134. The vector of claim 1 wherein the vector diagnostic sequence has at least 95% identity to the target diagnostic sequence.

135. The vector of claim 1, wherein after the vector and the target molecule are attached and the distance between the vector diagnostic sequence and target diagnostic sequence after attachment of the vector and the target molecule is less than 3000 bases.

136. The vector of claim 1, wherein after the vector and the target molecule are attached and the distance between the vector diagnostic sequence and target diagnostic sequence after attachment of the vector and the target molecule is less than 2000 bases.

137. The vector of claim 1, wherein after the vector and the target molecule are attached and the distance between the vector diagnostic sequence and target diagnostic sequence after attachment of the vector and the target molecule is less than 1000 bases.

138. The vector of claim 1, wherein after the vector and the target molecule are attached and the distance between the vector diagnostic sequence and target diagnostic sequence after attachment of the vector and the target molecule is less than 500 bases.

139. The vector of claim 1, wherein after the vector and the target molecule are attached and the distance between the vector diagnostic sequence and target diagnostic sequence after attachment of the vector and the target molecule is less than 300 bases.

140. The vector of claim 1, wherein after the vector and the target molecule are attached and the distance between the vector diagnostic sequence and target diagnostic sequence after attachment of the vector and the target molecule is less than 100 bases.

141. The vector of claim 1, wherein the vector is a TAR vector.

142. The vector of claim 1, wherein the vector further comprises a yeast centromere and a yeast telomere.

143. The vector of claim 142, wherein the vector further comprises an ARS.

144. A method of attaching two nucleic acid molecules together comprising mixing a target molecule and the vector of claim 1 together under conditions that promote the attachment of the target molecule and the vector of claim 1.

145. A product produced from the process of the method of claim 102.

147. A mixture comprising a vector comprising a marker sequence, a hook capable of attaching the vector to a target molecule, and a vector diagnostic sequence and a target molecule comprising a target diagnostic sequence, wherein the marker, hook, and vector diagnostic sequence are arranged 5′ to 3′ such that the marker would be in between the vector diagnostic sequence and the target diagnostic sequence after the vector and the target molecule are attached via the hook.


SEQUENCE LISTING (9 sequences)

SEQ ID NO: 1 - DNA, 148 bp, Artificial Sequence
  Description of Artificial Sequence/note = synthetic construct
ggattgccatcatggctggagcagagacatgaagcaagaaggccatggagatgagggcagggagatcccggagtggggagatcagatggggctctgtgtatcatgcaaaggactttgcattctgttccaagagctgggaaggttgaca

SEQ ID NO: 2 - DNA, 1001 bp, Artificial Sequence
  Description of Artificial Sequence/note = synthetic construct
aagcaccacaaagttagaggtcaagcaataatttggagaaaagaattagtaatttgttggacagacaaaagacttttttaatataacaaaaactttaaaaattaaaaaaatacacattcgaggacattttcctaaaaacacaggcaaaggacataaacagcaaagcaagaagacagcttgatgtggccattttatccagggggacattttggtgagccctatggacacagctgccatgatgccaacaatgtgacagctgtccccttcaaaatgcgttagccccagctcttcctctcccccaacctccagtccaaaggacttgcactttctactttactcctttctgcattgtttaattttcttttacaaatatgttacttgtcatcagaaaaaataaagaaataaataaactgttagagtgttagccccttaaaggggagcaagaatcacctttctaaaagaaagtttatgttaaatataatattagcatatgtgaatcctgagagaaaagttaacagtttagttgagttatttcctctgtagtctggagctaaaaatagggaatcttattctgtcctaaatcttttccttcctccacccagtgtctgtctggatcgaattcattcattcactcagtaggcactcactcagccaggcatggtgctaggcctcaggacctcgctgtgaaccagaaactgtccctacccccatggtgcaggcattctgcttgggagttggaggaggaacaggtaaaaaataattaaatattcaggttaacgatatattgtcaggtttgaggattgaggaaagggcgcagagagtggcaagggctgctgtttagatacagtggccaggaggctccgatgaggtgacctttgaggagagacatgcaggagatgaggggacagtgaagaggatttctaagaacactccaggcagacagaacagcgacagccaaggccctgaagtgggtaggggcctggtgtgtgtgaggaacctca

SEQ ID NO: 3 - DNA, 65 bp, Artificial Sequence
  Description of Artificial Sequence/note = synthetic construct
agcccttgccactctctgcgccctttcctcaatcctcaaacctgacaatatatcgttaacctgag

SEQ ID NO: 4 - DNA, 55 bp, Artificial Sequence
  Description of Artificial Sequence/note = synthetic construct
agcccttgccactctctgcgccctttcctcaatcctcaaacctgacaatatatcg

SEQ ID NO: 5 - DNA, 51 bp, Artificial Sequence
  Description of Artificial Sequence/note = synthetic construct
agcccttgccactctctgcgccctttcctcaatcctcaaacctgacaatat

SEQ ID NO: 6 - DNA, 47 bp, Artificial Sequence
  Description of Artificial Sequence/note = synthetic construct
agcccttgccactctctgcgccctttcctcaatcctcaaacctgaca

SEQ ID NO: 7 - DNA, 41 bp, Artificial Sequence
  Description of Artificial Sequence/note = synthetic construct
agcccttgccactctctgcgccctttcctcaatcctcaaac

SEQ ID NO: 8 - DNA, 26 bp, Artificial Sequence
  Description of Artificial Sequence/note = synthetic construct
agcccttgccactctctgcgcccttt

SEQ ID NO: 9 - DNA, 5 bp, Artificial Sequence
  Description of Artificial Sequence/note = synthetic construct
agccc